(19) United States
(12) Patent Application Publication
(10) Pub. No.: US 2003/0059951 A1
(43) Pub. Date: Mar. 27, 2003

(54) Maternal status testing in animals

(76) Inventors: Susan L.M. Frushour, Littleton, CO (US); Martin Pearson, Durham (GB); Edward Slowikowski, Durham (GB); Kevin D. Jones, Kent (GB)

Correspondence Address: SHERIDAN ROSS PC, 1560 BROADWAY, SUITE 1200, DENVER, CO, 80202

(21) Appl. No.: 10/219,605

(22) Filed: Aug. 14, 2002

Related U.S. Application Data
(60) Provisional application No. 60/312,274, filed on Aug. 14, 2001.

(51) Int. Cl. G01N 33/53

(52) U.S. Cl. 436/510

(57) ABSTRACT

Antibodies to maternal status factors are produced and isolated. They are used in a diagnostic assay to determine maternal status in cattle. The maternal status is evaluated using a test device which contains immobilized antibody to the bovine factor. In addition, a liquid sampling device which allows only a predetermined amount of biological liquid sample to contact the test device is provided.

### CROSS-REFERENCE TO RELATED APPLICATIONS

This application claims benefit under 35 USC § 119(e) of U.S. Provisional Application No. 60/312,274, filed Aug. 14, 2001, and under 35 USC § 120 copending U.S. patent application Ser. No. 09/516,848, filed Mar. 2, 2000, which are incorporated herein in their entirety by this reference.

### FIELD OF THE INVENTION

The present invention relates to antibodies directed to blood factors, a process for producing and isolating the same, and their use in rapid diagnostic assays for the evaluation of maternal status.

### BACKGROUND OF THE INVENTION

Reproductive efficiency is key to success in livestock industries. Recent advances have led to improving genetics, breeding, and disease control. Improvements in pregnancy detection have not kept pace in food animals and equine husbandry. The situation in cattle industries provides an example of how the present invention can benefit the livestock industries.

In today's ever increasingly competitive markets, the productivity of a cattle herd in terms of calves per cows must be optimized for maximum profitability. In order to achieve this, the time a cow is not bearing a calf must be minimized. Beef and dairy farming is an important part of the world food production industry in providing both milk and beef. There are approximately 34 million beef cows and 7 million dairy cows in the United States alone which contribute to the total world-wide number of 2 billion animals.

Those skilled in the art of animal reproduction report and accept the occurrence of a high (spontaneous) embryo loss. Knowing this, the term “pregnancy” or “maternal status,” from an animal husbandry and production aspect, means the presence of an embryo that with a high degree of probability will result in the birth of viable offspring.

One of the major problems in cattle breeding is the reduction of calves born from the failure of the heifer or cow to follow through the gestation cycle. Another major problem in cattle breeding is the time it takes to determine the maternal status of a cow. This delay in ascertaining the maternal status delays the time at which those cows that are not pregnant (i.e. that are “open”) can be identified and returned to breeding. The primary cause of this delay is the lack of diagnostic methods to determine the actual pregnancy results within a short time after breeding. The current technologies such as ultrasound, heat, or invasive examination by experienced veterinarians only work with any degree of confidence after 45 days of conception. An assay method that could reliably detect a successful pregnancy or “open” maternal status in the time period of 20 to 25 days post breeding or insemination would provide a significant savings to the ranch or dairy as opens could be recycled into the breeding program as quickly as possible thereby avoiding the extra costs associated with treating an open cow as a pregnant cow.

Factors affecting net calf crop in a disease-free beef herd that was naturally mated over 14 years were reviewed by Bellows et al. (1979). Four factors were identified that reduced net calf crop including non-pregnancy of females (17.4%), perinatal calf death (6.4%), calf deaths between birth and weaning (2.9%), and fetal deaths during gestation (2.3%). The primary cause of the delay in conception and pregnancy identification in the cow is lack of a diagnostic method to determine the actual results within a short time after breeding. An Australian study conducted in 1997 showed that following a single mating, the percentage of cows that produce a calf is only 55%. This 45% reduction was due largely to early embryonic mortality (EEM) occurring up to 24 days after fertilization and accounting for a loss of approximately 25% of pregnancies or late embryonic mortality, which occurs between 25 and 45 days following fertilization, and accounts for a further 14% loss.

The length of gestation in the cow, from the time of fertilization until actual birthing, of a calf ranges between 279-285 days with an average of 283 days. Therefore, 60 percent of the reduction in calves could be attributed to failure to mate, fertilization failure, and/or embryonic mortality. This is a very conservative estimate because beef cows could be mated two to three times during the 45 to 60 day exposure to bulls. Dairy cattle or artificially inseminated cattle are often “short cycled” to allow for maximum breeding opportunities.

Development of an easily used test offers an economic benefit for those in the breeding and beef industry in several ways. Such a test would allow the beef ranchers to rotate their cows to high quality bulls more quickly. In the artificial insemination industry (70% of cows in the United States are artificially inseminated—especially in the dairy industry), a rapid test would provide early results on cows that had been inseminated. This would allow the rancher to present his non-pregnant cows to the bull 22 days later or allow the rapid test to pick up non-pregnant cows 6-21 days after breeding, which can then be injected with prostaglandin to create a short cycle and be bred again 3 days later.

Maternal recognition must take place around day 16 or day 17, or the uterus will produce prostaglandin F2a causing regression of the corpus luteum and allow progesterone levels to drop preventing embryo implantation. Significant embryo losses can occur during this period due to either failure of the embryo to produce the signal or failure of the mother to recognize the signal from the embryo. In either one of these situations, the cow can be short cycled or allowed to return naturally to estrous in about 21 days.

The conventional method for determining pregnancy in cows involves a rectal palpitation which requires an experienced veterinarian or technician to detect pregnancy as early as 30 days after breeding or insemination. This method requires great care, much practice, and a well-developed sense of touch. Some experienced veterinarians can detect a pregnancy by 30 days but very few persons can reliably determine pregnancy using this method. The risk factor of bovine abortions is extremely high until day 20. Rectal palpitation is an invasive manual veterinary diagnostic method that is costly, requires a specialist and carries risk of peritonitis and death of the cow and calf.

Thus, the early determination of maternal status in bovines will significantly improve and provide greater efficiency in the production of bovines resulting in great economic benefit. Therefore, there is a need for a simple, rapid and accurate means for detecting pregnancy in bovines.

Another example of the importance of determining mating success in livestock industries is in racehorse breeding. Correctly timed successful mating contributes to the maturity of the offspring as it progresses through the various chronological age groups used in racing. Pregnancy testing will greatly assist in recycling mares that have not been successfully bred to achieve pregnancy at the earliest possible time.

### SUMMARY OF THE INVENTION

There are two approaches that routinely have been employed to select analytes for use in pregnancy tests: (1) Detect analytes produced by the conceptus/embryo/fetus including the placenta, a fetal membrane and (2) detect analytes produced by the maternal animal in response to the presence of pregnancy. While the first approach for tests for analytes have served as the basis for useful tests for human pregnancy, attempts to employ this approach for domestic animals have not been successful to date. Alternatively, some of the classic animal pregnancy tests in bovines (estrone and progesterone) and equines (PMSG) have employed the second approach of detecting analytes produced by the maternal animal with limited commercial success.

To overcome the unsatisfactory outcome of these efforts, the present inventors arrived at the novel process of producing antibodies against analytes present in body fluids, such as blood or blood fraction, of pregnant animals and removing antibodies that are reactive with substances in non-pregnant animals. The selected antibodies are then used to prepare tests to determine pregnancy in female animals. A further object of the invention is to use analyte(s) present in the bodily fluids of a pregnant animal that are most reliably indicative of pregnancy, regardless of their origin or composition providing that they are antigen and react with antibodies specific to their composition.

The present inventors have developed immonoassays which detect specific markers (i.e., factors) present in saliva, milk, sera, blood, or other body fluids indicative of maternal status. By using the assay method of the present invention, pregnancy detection as early as 15 days after insemination (as opposed to the current 40-45 days) is feasible.

The present invention provides a method for producing antibodies to factors indicating conception and pregnancy (i.e., pregnancy factors) and its use in determining maternal status in any animal producing pregnancy factors after conception.

### BRIEF DESCRIPTION OF THE DRAWINGS

FIG. 1 is a schematic representation of a test device for use in an assay test in accordance with the invention.

FIG. 2 is an illustration of the test device of FIG. 1 showing the direction of liquid sample flow in accordance with the invention.

FIG. 3 illustrates a variety of detection zone markers for a possible positive pregnancy test indication.

FIG. 4 shows one embodiment of the top portion of a liquid sampling tube of the present invention for use with a test device.

FIG. 5 shows one embodiment of a body portion of a liquid sampling tube of the present invention.

FIG. 6 shows one embodiment of a bottom portion of a liquid sampling tube of the present invention.

FIG. 7 shows an exploded view of a preferred embodiment of a testing device.

### DETAILED DESCRIPTION OF THE INVENTION

The present invention provides a method for producing antibodies to factors indicating conception and pregnancy (i.e., pregnancy factors) and its use in determining maternal status in any animal producing pregnancy factors after conception. While not wishing to be bound by any such representation, it is believed that, in addition to bovines, animals which produce pregnancy factor include, without limitation; humans, domestic animals such as swine, sheep and goats; and other mammals such as rabbits, cats and dogs. Although the present invention will now be described in reference to maternal status evaluation in a bovine, it should be appreciated that the invention is not limited to bovine subjects. As used in this invention, the term “antibody” refers to an immunoglobulin which binds to a specific antigen or hapten. The term “factors indicating conception and pregnancy” or “pregnancy factors” refers to a compound, for example, a protein or a hormone, that is present in the blood stream and is produced by a pregnant cow. It should be understood that the pregnancy factor may include more than one type of compound. However, for the sake of brevity and clarity, both plural and singular forms of the pregnancy factor and the corresponding antibodies will be referred to in a singular form. Thus, the preferred antibody of the present invention is a polyclonal antibody although monoclonal antibodies and antigen binding fragments are also contemplated for the invention. The antibody of the present invention is produced by obtaining biological fluids from a cow in its early pregnancy, injecting the biological fluid into another animal which is capable of producing antibody to the biological fluid, isolating antibodies raised against the biological fluid and substantially removing antibodies that are negative to the factor of early pregnancy. It will be understood by those skilled in the art that this approach will be applicable to any animal species and at any selected stage of pregnancy.

The production of an antibody in response to a given antigen or hapten is a common occurrence in humans and animals. The antibody is polyclonal in mammals however, monoclonal antibodies can also be produced, for example, by using carcinoma cell lines. Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the desired antigen(s), serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

The antibodies of the present invention can be prepared by taking a biological fluid sample from an animal that is less than 100 days pregnant, preferably from about 20 days to about 40 days pregnant, more preferably from about 20 to about 30 days pregnant.

The term “biological fluid” refers to any fluid that can be obtained from the bovine. Examples of such fluids include blood, saliva, urine, milk, perspiration and chorionic fluid. Preferably, the biological fluid is blood. For the production of the antibody, the acellular fraction is separated from the cellular fraction by any known method such as by centrifuge, settling, or filtration. The acellular fraction, i.e., serum, is then mixed with Freunds complete adjuvant and injected into a non-bovine animal following a pre-immunization test bleed. Exemplary non-bovine animals useful in generating antibodies to the factor of early pregnancy include mammals such as sheep, goats, equines, swine, mice, rabbits and poultry. In addition, an egg can also be used to produce an antibody, e.g., IgY, for the factor of early pregnancy. A second injection of the acellular fraction in Freunds Incomplete adjuvant is injected into the same animal 21 days after the first injection was performed. A third injection of the acellular fraction in Freunds Incomplete adjuvant is injected into the same animal 42 days after the first injection was performed. The antibody is isolated from the non-bovine animal via a production bleed performed at the end of a suitable incubation period. Typically, the incubation period is from about 10 days to about 50 days, preferably from about 20 days to about 50 days, and more preferably from about 25 days to about 50 days, measured from the day the first injection was performed. Isolation of the antibody of the present invention generally involves obtaining antibodies present in the blood or the lymph node system of the immunized animal and isolating the antibody to the bovine factor of early pregnancy. For example, in cases where a goat is used to generate the antibody, the goat is incubated with the serum containing bovine pregnancy factor for about 6 weeks to about 2 months.

The following discussion describes a method of production of an antibody in a preferred production animal, a rabbit. However, one of skill in the art will be able to adapt this protocol to other animals. In addition, minor variations in injection timepoints to optimize antibody production and collection are contemplated. In the preferred case, a rabbit is used to generate the antibody and the serum containing the bovine pregnancy factor is either used without prior purification or is first partially purified before injection. Initial purification methods may include passing the serum over an affinity column containing goat anti-bovine IgG, and/or ammonium sulfate precipitation. The bovine serum containing the bovine pregnancy factor or the partially purified bovine pregnancy antigen is then injected into the rabbit. The rabbit is then re-injected at about 21 and about 42 days after the initial injection. Blood may be recovered from the rabbit at about 30 days after the initial injection to test for the presence of anti-bovine pregnancy factor antibodies. The blood from the rabbits is then collected at about 50 days after the initial injection. Antibodies, e.g., immunoglobulin G (i.e., IgG), are then purified by Protein A affinity chromatography from the goat or rabbit blood obtained. IgG is further purified to remove the majority of the non-pregnancy factor antibodies by passing through a column that contains immobilized normal, i.e., non-pregnant, bovine serum. The purity of the antibody can be further increased by passing the resulting antibody through another column containing immobilized non-pregnant bovine serum. The antibody thus obtained selectively binds to proteins present in the serum of pregnant cattle. These factors appear in the blood of a pregnant cow as early as 5 days following fertilization. These proteins are detectable between 20 and 50 days following fertilization.

According to the present invention, the phrase “selectively binds to” refers to the ability of an antibody of the present invention to preferentially bind to specified proteins (e.g., a bovine pregnancy factor protein). More specifically, the phrase “selectively binds” refers to the specific binding of one protein to another (e.g., an antibody to an antigen), wherein the level of binding, as measured by any standard assay (e.g., an immunoassay), is statistically significantly higher (p<0.05) than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc. The purity of the antibody of the present invention is at least about 30% by weight (by wt.), preferably at least about 60% by wt., and more preferably at least about 90% by wt. The antibody is stored in PBS buffer, pH 7.4 containing 0.1% sodium azide, at 2-8° C.

Antibodies used in practice of the present invention may be prepared by partially purifying the animal serum used in immunizing the host animal to remove contaminating antigens, removing antibody contaminants from the antibody preparations, or by both means. For example, antibodies to normal, non-pregnant animal serum are produced and immobilized by any of various methods available. Body fluid, such as serum, from pregnant animals is treated with the water-insoluble antibodies against substances present in non-pregnant animals until such substances are removed. The remaining pregnancy related substances are injected into host animals for the purpose of producing antibodies. In addition, contaminating antibodies, that is those that bind to substances not associated specifically with pregnancy, can be advantageously removed by treating antibody preparations produced against either intact or partially purified mixtures of substances associated with pregnancy with non-pregnancy associated substances suitably immobilized. In some instances it may be particularly preferred to use both partial purification of the antigen as well as removing contaminating antibodies to prepare materials for the present invention.

Monoclonal antibodies to the pregnancy factors can also be prepared using a process similar to that discussed by Milstein and Kohler as reported in Nature, 1975, 256, 495-497. Generally, the preparation of a monoclonal antibody involves injecting a mouse (or other suitable animal) with partially or completely purified bovine pregnancy factor. The immunized animals are sacrificed and the cells from their spleens are fused, e.g., with mouse myeloma cells. The result is a hybrid cell, known as a “hybridoma” that is capable of reproducing in vitro. The population of hybridomas is then screened for immunoglobulin production using any of the known methods, for example, as described in U.S. Pat. No. 4,016,043. The immunoglobulins present in the cell culture fluids are then further examined for their ability to react with the bovine pregnancy factor used for immunization.

Antibodies prepared by the methods described above or other methods equally satisfactory are used in various immunoassay methods well-known in the art including but not limited to: ELISA, radioimmunoassay, particle agglutination assay, immunoturbidimetric assay, immunochromatographic assay, and others. It is a particular advantage of the present invention that antibodies produced against pregnancy-specific substances can be employed in test methods that meet the needs of users. For example, it is an advantage to ranchers to be able to test pregnancy with a single disposable test device such as that made possible by immunochromatographic technologies. Alternatively, it may be preferred on large farms with laboratory facilities to test for pregnancy of the many animals using an automated instrument. The inventors envisioned these and other test methods for the present invention.

In one particular embodiment, the test device includes a dry porous solid phase with immobilized antibody in a detection zone. The use of a solid phase assay with a visible change indicative of the presence or absence of a given substrate is generally described in U.S. Pat. Nos. 4,703,017, and 5,656,503 which are incorporated herein in their entirety by this reference.

The porous solid phase comprises any material, or combination of materials, having a high permeability to liquids. Exemplary porous solid phase materials include porous plastic materials such as polypropylene, polyethylene (preferably of very high molecular weight), polyvinylidene fluoride, ethylene vinyl acetate, acrylonitrile and polytetrafluoroethylene; glass fibers; paper; cellulosic materials such as nitrocellulose; cellulose esters such as cellulose acetate; Nylon®; Rayon®; polyester; polyethersulfone; and other suitable materials known in the art or combinations thereof. It can be advantageous to pre-treat the porous solid phase material with a surface-active agent during manufacture, as this can reduce any inherent hydrophobicity in the porous solid phase material thereby enhancing its ability to take up and deliver a moist sample efficiently. Preferably, the porous solid phase material includes a nitrocellulose sheet having a pore size of between 0.1 micron and 20 microns, more preferably between 3 microns and 12 microns, and most preferably between 5 microns and 8 microns. The preferred membrane is Immunopore FP7 (Whatman).

The antibody may be bound to the matrix of the detection zone by any one of a number of methods known to the art. For example, the antibody may be absorbed onto various water insoluble matrices such as microtiter plates, Dextran beads, nylon web, glass, cellulose, polyacrylamide, charcoal, urethane, ceramic, or mixtures thereof; chemically bonded to the porous solid phase material, i.e., by the formation of ionic or covalent bonds, including disulfide bond formation, hydrogen bonding, Van der Waals bonding and charge attraction; or physically attached to the porous solid phase material, i.e., by absorption, entrapment in an insoluble matrix; and the like. The bound antibody can then be provided in a kit wherein body fluids from female bovine animals would be added and activity of the antibody with the fluids could be measured.

The antibody in the detection zone is permanently immobilized in the detection zone on the porous solid phase material and is therefore not mobile in the moist state. The relative positioning of the labeled antibody, if present within the test device, and the detection zone are such that a liquid sample which is applied to the device can pick up a labeled antibody (either from the liquid sample or the labeled antibody zone) and thereafter permeate into the detection zone. The antibody in the detection zone can be immobilized firmly with or without prior chemical treatment. For example, if the material comprising the porous solid phase is nitrocellulose, which is preferred, then the antibody can be immobilized firmly by applying a liquid solution containing the antibody and allowing it to dry. It is to be understood that the term “nitrocellulose” refers to nitric acid esters of cellulose, which may be nitrocellulose alone, or a mixed ester of nitric acid and other acids, and in particular aliphatic carboxylic acids having from one to seven carbon atoms, with acetic acid being preferred. Such solid supports which are formed from cellulose esterified with nitric acid alone, or a mixture of nitric acid, are often referred to as nitrocellulose paper.

Although nitrocellulose is a preferred porous solid phase material, it is to be understood that other materials, having a surface area sufficient for supporting the antibody in a concentration as hereinafter described may also be employed for producing such porous solid phase.

In general, the porous solid phase which is used in the assay has a surface area such that it is capable of supporting antibody in a concentration of at least about 0.2 mM/cm², and preferably in a concentration of at least about 1 mM/cm².

Preferably, the immobilized antibody in the detection zone is impregnated throughout the thickness of the porous solid phase material in the detection zone (e.g., throughout the thickness of the sheet or strip if the porous solid phase material is in this form). Such impregnation can enhance the extent to which the immobilized antibody can capture any labeled antibody-pregnancy factor complex present in the migrating sample.

The liquid solution useful in applying the antibody to the detection zone for immobilization can include a buffer solution. Useful buffer solutions include any buffer solution having a pH from about pH 6.5 to about pH 9.5. Exemplary useful buffer solutions include phosphate buffers with a phosphate level in the range of from about 10 mM to about 100 mM, phosphate buffered saline buffers, TRIS buffer pH 8.2 in the range of from about 10 mM to about 50 mM, TRIS buffered saline pH 8.2, acetate buffer, carbonate buffer, bicarbonate buffer, MOPS piperazine buffer, BIS-TRIS buffer, tricine buffer, HEPES buffer and the like.

The liquid solution can also include a surfactant. Any general surfactant can be used. Exemplary surfactants useful for the present invention include Tween 20⁷, Triton X-100⁷. The amount of surfactant can be from about 0.01% to about 10%, preferably about 0.5%.

If the porous solid phase material comprises paper, the immobilization of the antibody in the detection zone needs to be performed by covalent linkage which can be achieved through chemical coupling using, for example, aldehydes, azo compounds, carboxylic acids, isothiocyanates, cyano compounds, CNBr, carbonyldiimidazole, or tresyl chloride.

Following the application of the antibody to the detection zone, the remainder of the porous solid phase material may be treated to block any remaining binding sites. Blocking can be achieved by treatment with protein (e.g., bovine serum albumin or milk protein), with polyvinyl alcohol or ethanolamine, surfactant, polymers (such as PVA, PVP, PEG), or combinations thereof.

The test device can also include a sample receiving zone for applying a sample, e.g., a liquid biological sample such as urine, serum, or saliva. The porous solid phase material in the sample receiving zone can include any bibulous, porous or fibrous material capable of absorbing liquid rapidly. The porosity of the material can be unidirectional (i.e., with pores or fibers running wholly or predominantly parallel to an axis of the member) or multidirectional (omnidirectional, so that the member has an amorphous sponge-like structure). Preferably the porous solid phase material of the sample receiving zone should be chosen such that the sample receiving zone can be saturated with aqueous liquid within a matter of seconds. Preferably the material remains robust when moist. The liquid must thereafter permeate freely from the porous sample receiving zone into the remaining porous solid phase material of the test device. Although any of the above listed porous solid phase materials can be used in the sample receiving zone, use of glass fiber in the sample receiving zone is preferred.

The liquid biological sample applied to the sample receiving zone may include serum or whole blood. Whole blood is the preferred biological sample to collect for testing. When whole blood is the biological fluid to be tested, the introduction of a blood separation filter between the sample receiving zone and the conjugate release pad will allow whole blood samples to be used directly without needing an additional serum-separation step. Examples of suitable blood separation filters for use in a whole blood testing device include Whatman F147-11, GFA/VA or GFD/VA membranes.

The test device can also include a labeled bovine pregnancy factor antibody zone (“labelled antibody zone”) within the porous solid phase located downstream relative to the sample receiving zone but upstream relative to the detection zone. As used in this invention, the term “labelled antibody” refers to antibody which is bound to a label. The label can be any entity, the presence of which can be readily detected by any of the known methods in the art including visual inspection, using an instrument, conducting a chemical reaction and combinations thereof. The term “visual” as used herein means that the results of the test can be seen without the use of instrumentation, i.e., with the naked eye. Preferably the label is a direct label, i.e., an entity which, in its natural state, is readily visible either to the naked eye, or with the aid of an optical filter and/or applied stimulation, e.g., UV light to promote fluorescence. For example, minute colored particles, such as dye sols; metallic sols such as gold, platinum, palladium, iron, copper; colored latex particles; and carbon sols are all suitable. Of these metallic sol particles are preferred with gold sol particles being particularly preferred. The term “sol particle” refers to particles of a sol. And the term “metal sol particle” refers to particles of a sol consisting of a metal, a metal compound or polymer nuclei coated with a metal or metal compound. The useful size of a sol particle depends on the particular sol particle being used, for example, for gold sols, useful sizes range from about 15 nm to about 100 nm, with about 40 nm being particularly preferred. The production of colloidal gold is based upon standard colloid growth techniques. The basic method includes mixing a reducer and a gold salt together in such a way that there is relatively gentle reduction resulting in a slow and reproducible particle growth. Using metallic and non-metallic sol particles for immunoassay is generally described in U.S. Pat. Nos. 4,313,734 and 4,954,452, respectively, which are incorporated by reference herein in their entirety. Concentration of the label into a small zone or volume should give rise to a readily detectable signal, e.g. a strongly colored area. This can be evaluated by eye, or by using instruments if desired.

Indirect labels, such as enzymes (e.g., alkaline phosphatase and horseradish peroxidase), radio isotopes, fluorescent compounds or other compounds, which can be detected using appropriate instruments, can also be used but these usually require an instrument or the addition of one or more developing reagents such as substrates before a visible signal can be detected. Such additional reagents can be incorporated in the porous solid phase material or in the sample receiving zone, such that they dissolve or disperse in the aqueous liquid sample. Alternatively, the developing reagents can be added to the sample before contact with the porous solid phase material or the porous solid phase material can be exposed to the developing reagents after the binding reaction has taken place.

Coupling of the label to the specific binding reagent can be by covalent bonding, if desired, or by hydrophobic or electrostatic bonding. Such techniques are well known to one of ordinary skill in the art. In general, the porous solid phase material which is used in the assay has a surface area such that is capable of supporting labeled antibody in a concentration of at least about 0.2 mM/cm², and preferably in a concentration of at least about 0.5 mM/cm².

The selection of one or more suitable labeled antibodies on the porous solid phase material is deemed to be within the scope of those skilled in the art from the teachings herein.

To assist the free mobility of the labeled antibody when the porous solid phase is moistened with the sample, it is preferable for the labelled antibody to be applied to the porous solid phase as a surface layer, rather than being impregnated in the thickness of the porous solid phase material. This can minimize interaction between the porous solid phase material and the labeled antibody. In one particular embodiment of the present invention, the porous solid phase material is pre-treated with a glazing material in the region to which the labeled antibody is to be applied. Glazing can be achieved, for example, by depositing an aqueous sugar or cellulose solution, e.g., sucrose or lactose, on the porous solid phase at the relevant portion, and drying. The labeled antibody can then be applied to the glazed portion. The remainder of the porous solid phase should not be glazed.

Alternatively, the labeled antibody can be provided separately from the test device and admixed with the sample prior to being applied to the sample receiving zone of the test device. In this manner, any bovine pregnancy factor which may be present in the sample is allowed to bind with the labeled antibody prior to, or during application of the sample to the sample receiving zone. Such labeled antibody can be provided as a solution or a solid which is then admixed with the sample. When the labeled antibody is in a solution, the solution generally comprises a buffer solution which stabilizes the labeled antibody, i.e., prevents a significant decrease in the antibody activity by, e.g., preventing decomposition or denaturing of the antibody. Useful buffer solutions include those described above. The solution may also include a surfactant described above.

Whether the labeled antibody is an integral part of the test device or is provided separately, it is essential that the labeled antibody migrates with the liquid sample as this progresses to the detection zone. Preferably, the flow of the sample continues beyond the detection zone and sufficient sample is applied to the porous solid phase material in order that this may occur and that any excess labeled antibody which does not participate in any binding reaction in the detection zone is flushed away from the detection zone by this continuing flow. If desired, an absorbent “sink” can be provided at the distal end of the porous solid phase material. The absorbent sink may comprise, for example, Whatman 3MM chromatography paper or Whatman 17 Chr, and should provide sufficient absorptive capacity to allow any unbound labeled antibody, i.e, conjugate, to wash out of the detection zone. As an alternative to such a sink it can be sufficient to have a length of porous solid phase material which extends beyond the detection zone.

The test device employed in the assay is preferably in sheet form, generally being in the form of a card, a test strip, a flow-through device or dipstick, etc. It should be appreciated, however, that other forms of test devices are also within the spirit and scope of the invention. The antibody and the labeled antibody, if present, are applied in spatially distinct zones of the test device, and the liquid sample is allowed to permeate through the test device from one side or end to another. Generally, the porous solid phase of the test device is capable of conveying a liquid sample in a fluid flow direction parallel to the length of the test device.

The spatial separation between the zones (i.e., sample receiving zone, labeled antibody zone, if used, and the detection zone), and the flow rate characteristics of the porous solid phase material can be selected to allow adequate reaction times during which the necessary specific binding can occur, and to allow the labeled antibody in the labeled antibody zone to dissolve or disperse in the liquid sample and migrate through the porous solid phase material. Further control over these parameters can be achieved by the incorporation of viscosity modifiers (e.g., sugars and modified celluloses) in the sample to slow down the reagent migration.

Preferably, the porous solid phase material is “backed”, e.g., with plastic sheet, to increase its handling strength. This can be manufactured easily by forming a thin layer of porous solid phase material such as nitrocellulose on a sheet of backing material. The actual pore size of the porous solid phase material when backed in this manner may be lower than that of the corresponding unbacked material. Alternatively, a pre-formed sheet of porous solid phase material can be tightly sandwiched between two supporting sheets of solid materials, e.g., between two supporting plastic sheets.

The antibody and/or the labeled antibody can be applied to the porous solid phase material in a variety of ways. Various “printing” techniques have previously been proposed for application of liquid solutions of such reagents to porous solid phase materials, e.g., micro-syringes, pens using metered pumps, direct printing and ink-jet printing, and any of these techniques can be used in the present invention. To facilitate manufacture, the porous solid phase material (e.g., sheet) can be treated with these reagents and then subdivided into smaller portions (e.g., small narrow strips each embodying the required reagent containing zones) to provide a plurality of identical porous solid phase material units.

The sensitivity of the assay, i.e., bovine pregnancy testing, can be increased by increasing the concentration of the labeled antibody and/or the antibodies on the detection zone. Accordingly, porous solid phase materials having high surface areas are particularly preferred in that the labeled antibody and/or the antibodies in the detection zone may be supported on such material in a high concentration. It should be appreciated, however, that the concentration of the labeled antibody and/or the antibodies in the detection zone which is actually used is dependent in part on the binding affinity of the labeled antibody and/or the antibodies in the detection zone. Therefore, the scope of the present invention is not limited to a particular concentration of labeled antibody and/or the antibodies on the detection zone.

Pregnancy testing of a female bovine generally involves contacting the test device as set forth above with an aqueous liquid sample (e.g., liquid biological sample such as whole blood, serum, saliva, urine or chorionic fluid) of the female bovine. A preferred aqueous liquid sample is whole blood. The sample is allowed to permeate by capillary action through the porous solid phase material via the labeled antibody zone, if present, migrates from the labeled antibody zone to the detection zone and the labeled antibody migrates therewith from the solution or from the labeled antibody zone, depending on how the labeled antibody is presented in the test. Thus, the labeled antibodies are carried downstream as the liquid sample front is moved along the test device by a capillary action. As the liquid sample moves along the length of the test device, any pregnancy factor that may be present in the liquid sample binds to the labeled antibody and the resulting complex is carried along the length of the test device to the detection zone. The presence of a factor of early pregnancy in the sample can be determined by observing the extent (if any) to which the complex becomes bound in the detection zone. The labeled antibody zone, if present in the test device, is located at a height sufficiently high enough in the test device such that when the liquid sample is applied, the labeled antibody zone is above the liquid sample level to avoid having the labeled antibodies diffusing into the liquid sample itself.

When the test device includes both the labeled antibody zone and the detection zone, the distance between these two zones should be sufficiently large enough to allow the formation of labeled antibody-pregnancy factor complex prior to the capture of the complex by the antibodies present in the detection zone. Preferably such a distance is at least about 1 cm, and more preferably at least about 2 cm.

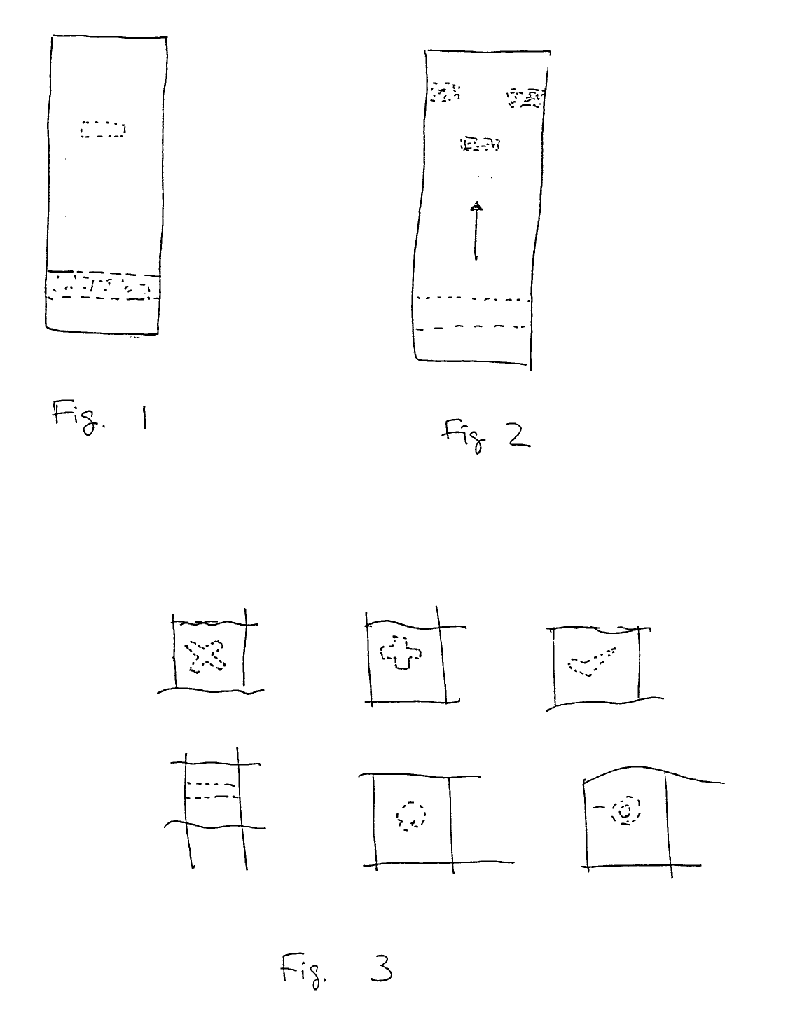

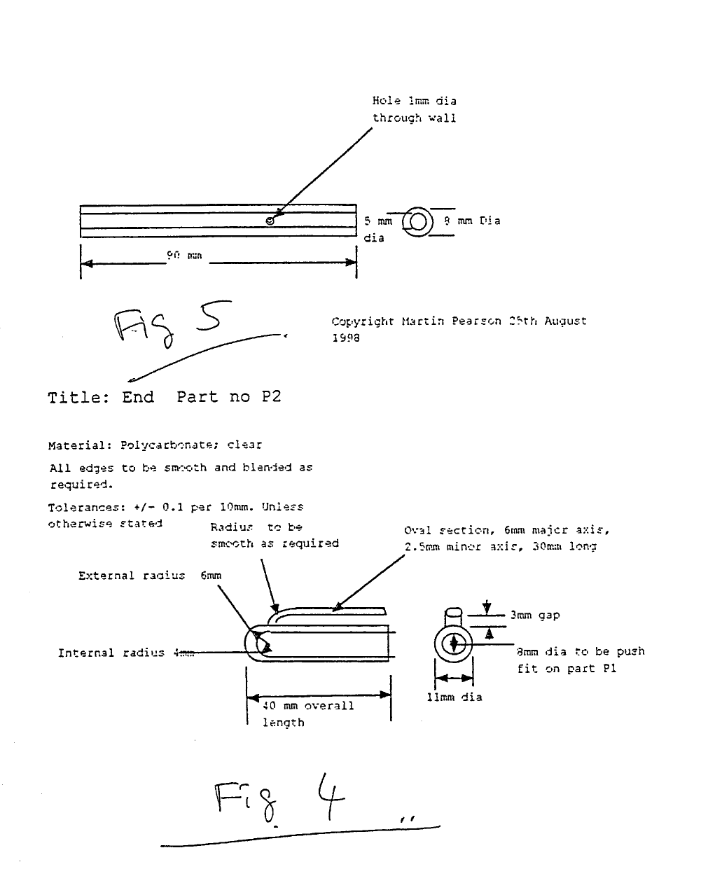

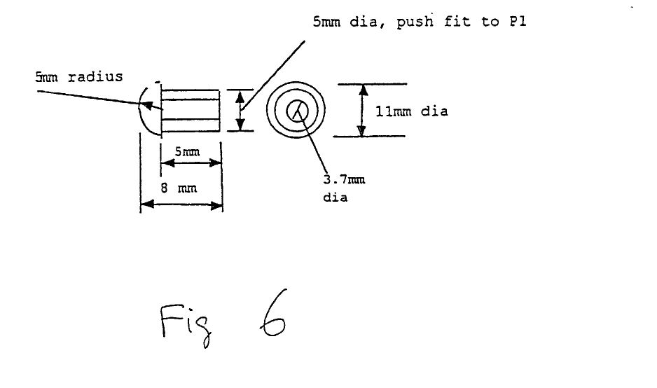

The detection zone can be any shape which aids in visualizing the bovine pregnancy test result. For example, as shown in FIG. 1, the detection zone can be a strip or line across the partial or entire width of the test device, an “x”, “+”, a check mark, a circle, or a ring.

Preferably, the observation is made visually by the presence of a particular color within the detection zone where the antibody has been immobilized. For example, with gold sols as labels the color red indicates the positive result, with carbon or palladium sols the positive pregnancy is indicated by black color, and a wide variety of color can be used as indicator for latex particles depending on the dye that is used.

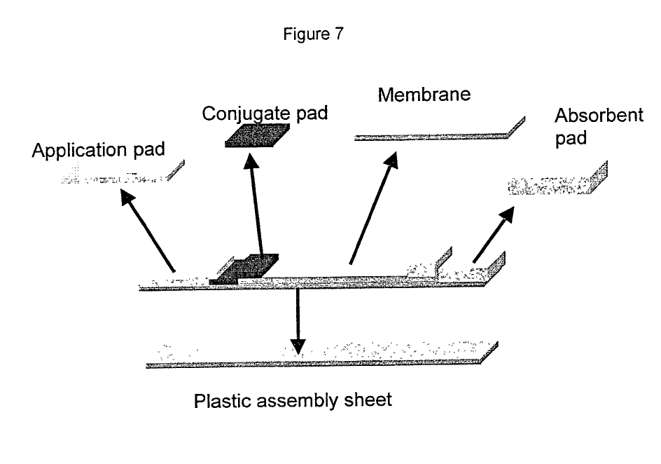

A preferred embodiment of the present invention is shown in FIG. 7. An application pad, a conjugate pad, a membrane serving as the detection zone and an absorbent pad are aligned linearly along, and attached to, a plastic assembly sheet. The moist biological sample is applied to the application pad and is drawn by capillary action across the conjugate pad and membrane towards the absorbent pad. Labeled antibodies are encountered in the conjugate pad and will bind to the specific maternal status protein factors, if present, in the biological sample as it moves across the conjugate pad. Preferably, the conjugate pad is Rapid 27Q (Whatman). As the sample is drawn across the membrane, immobilized anti-bovine antibodies are encountered. These immobilized antibodies may be rabbit anti-bovine, goat anti-bovine or anti-bovine antibodies of any other animal origin depending on the host animal used to generate the antibodies. For monoclonal antibodies, these antibodies would be mouse anti-bovine. These immobilized antibodies will bind to the labeled antibody migrating under capillary action towards the absorbent pad. The presence of the labeled antibody bound to the maternal status proteins is visualized on the membrane, in the detection zone, due to the presence of the label. In one embodiment, the antibody preparation present on the conjugate pad is preparation A, the preparation of which is described above. Similarly, in other embodiments, the antibodies present are preparations B, C or D. In another embodiment, the antibodies present comprise any combination of the preparations of A, B, C and D. When the A and D antibodies are combined in the test of the present invention, the accuracy of maternal status prediction is about 84.6%. When the B and C antibodies are combined in the test of the present invention, the accuracy of maternal status prediction is about 61.1%.

Pregnancy status may also be determined by any of various agglutination immunoassays know in the art including but not limited to erythrocyte and latex agglutination. In these methods, the antibodies of the present invention are labeled with red blood cells or synthetic latex particles respectively by physical, chemical, or immunochemical means. The labeled antibodies are allowed to interact with pregnancy-associated substances in the sample with mixing. Erythrocytes or latex particles in this mixture are caused to agglutinate by immunochemical interactions between the particle-bound antibodies and the substances to which the antibodies specifically bind. Pregnancy status is detecting by detecting agglutination of the particles, such detection may be accomplished by visual, optical, electrical, physical or other means practiced by those skilled in the art.

### EXAMPLES

The following Examples are provided to illustrate embodiments of the present invention and are not intended to limit the scope of the invention as set forth in the claims.

### Example 1

Using the general outline above, four functional antibody preparations (denoted “A” through “D”) have been prepared by different means that show different functionality in testing to evaluate the maternal status of cows. Preparation A is prepared by isolating serum from a cow that is 20-40 days pregnant. This serum is purified over a goat anti-bovine IgG affinity column to remove all bovine proteins not associated with pregnancy. The eluant of this purification column is injected into a non-bovine animal, preferably a rabbit. A small amount of blood (e.g. 3-5 ml) is collected from the rabbit prior to the injection of the purified serum. The rabbit is injected with one milliliter of the purified serum mixed with Complete Freunds adjuvant. The rabbit is then given another injection of the purified serum 14-30 days after the initial injection of the purified serum. Preferably, the rabbit is given this second injection 21 days after the initial injection of the purified serum. This second injection contains about 1 ml of the purified serum mixed with Incomplete Freunds adjuvant. Optionally, a test bleed is conducted on the rabbit at about day 30 following the initial injection date. At about day 42 following the initial injection date, the rabbit is given a third injection. Like the second injection, this third injection also contains about 1 ml of the purified serum mixed with Incomplete Freunds adjuvant. Blood is drawn from the rabbit between about 45 and about 75 days after the initial injection. Preferably, the blood is drawn from the rabbit about 50 days after the initial injection. The antibody is enriched from the serum of the collected blood by Protein A precipitation. The resulting A antibody preparation showed a 91% accuracy rate in the determination of maternal status of a female bovine when used in the testing procedures described below.

Preparation B is prepared by isolating serum from a cow that is 20-40 days pregnant. This serum is purified by ammonium sulfate precipitation to remove many nonspecific proteins not associated with pregnancy. The resulting partially-purified serum is injected into a non-bovine animal, preferably a rabbit. A small amount of blood (e.g. 3-5 ml) is collected from the rabbit prior to the injection of the partially purified serum. The rabbit is injected with one milliliter of the partially purified serum mixed with Complete Freunds adjuvant. The rabbit is then given another injection of the partially purified serum 14-30 days after the initial injection of the partially purified serum. Preferably, the rabbit is given this second injection 21 days after the initial injection of the partially purified serum. This second injection contains 1 ml of the partially purified serum mixed with Incomplete Freunds adjuvant. Optionally, a test bleed is conducted on the rabbit at about day 30 following the initial injection date. At about day 42 following the initial injection date, the rabbit is given a third injection. Like the second injection, this third injection also contains 1 ml of the partially purified serum mixed with Incomplete Freunds adjuvant. Blood is drawn from the rabbit between about 45 and about 75 days after the initial injection. Preferably, the blood is drawn from the rabbit about 50 days after the initial injection. The antibody is enriched from the serum of the collected blood by Protein A precipitation. The resulting B antibody preparation showed an 83% accuracy rate in the determination of maternal status of a female bovine when used in the testing procedures described below.

Preparation C is prepared by isolating serum from a cow that is 20-40 days pregnant. The serum is injected into a non-bovine animal, preferably a rabbit, without further purification. A small amount of blood (e.g. 3-5 ml) is collected from the rabbit prior to the injection of the purified serum. The rabbit is injected with one milliliter of the serum mixed with Complete Freunds adjuvant. The rabbit is then given another injection of the serum 14-30 days after the initial injection of the serum. Preferably, the rabbit is given this second injection 21 days after the initial injection of the serum. This second injection contains 1 ml of the serum mixed with Incomplete Freunds adjuvant. Optionally, a test bleed is conducted on the rabbit at about day 30 following the initial injection date. At about day 42 following the initial injection date, the rabbit is given a third injection. Like the second injection, this third injection also contains 1 ml of the serum mixed with Incomplete Freunds adjuvant. Blood is drawn from the rabbit between about 45 and about 75 days after the initial injection. Preferably, the blood is drawn from the rabbit about 50 days after the initial injection. The antibody is enriched from the serum of the collected blood by Protein A precipitation. The resulting C antibody preparation showed an 87% accuracy rate in the determination of maternal status of a female bovine when used in the testing procedures described below.

Preparation D is prepared by isolating serum from a cow that is 20-40 days pregnant. One-half of this serum is purified over a goat anti-bovine IgG affinity column to remove all bovine proteins not associated with pregnancy. The other half of this serum is purified using ammonium sulfate purification. The two halves are then mixed to produce the purified serum for injection in the animal to produce the antibodies. This purified serum is injected into a non-bovine animal, preferably a rabbit. A small amount of blood (e.g. 3-5 ml) is collected from the rabbit prior to the injection of the purified serum. The rabbit is injected with one milliliter of the purified serum mixed with Complete Freunds adjuvant. The rabbit is then given another injection of the purified serum 14-30 days after the initial injection of the purified serum. Preferably, the rabbit is given this second injection 21 days after the initial injection of the purified serum. This second injection contains 1 ml of the purified serum mixed with Incomplete Freunds adjuvant. Optionally, a test bleed is conducted on the rabbit at about day 30 following the initial injection date. At about day 42 following the initial injection date, the rabbit is given a third injection. Like the second injection, this third injection also contains 1 ml of the purified serum mixed with Incomplete Freunds adjuvant. Blood is drawn from the rabbit between about 45 and about 75 days after the initial injection. Preferably, the blood is drawn from the rabbit about 50 days after the initial injection. The antibody is enriched from the serum of the collected blood by Protein A precipitation. The resulting D antibody preparation showed a 93% accuracy rate in the determination of maternal status of a female bovine when used in the testing procedures described below.

### Example 2

This example describes the production of different bovine maternal status antibody preparations suitable for use in the present invention. These antibodies are produced in rabbits and the preparations are distinct, one from the other, as a result of the means by which the antigen injected into the rabbits is prepared.

Using Monoject Blood collection needles #216, 27 samples of blood were taken from the tail head area of dairy (Jersey) cows, possibly impregnated via artificial insemination, at various days into pregnancy starting at day 31. These were individually centrifuged to separate the hemoglobin, and the resulting serum was individually labeled. All samples were immediately frozen awaiting palpitation results done at 55 days from artificial insemination service. From these 27 tubes of frozen, spun sera, only 12 were confirmed pregnant by palpation. Six of these tubes were then chosen and shipped to the laboratory for use in antibody production.

Twelve New Zealand and/or California White rabbits for laboratory use were ordered from Lampire Biologicals, Box 270, Pipersville Pa. 18947. These rabbits were mature, fully developed and deemed healthy and free of any known diseases. IACUC required practices were used in handling the host animals.

Using Lampires Expressline Service, the frozen bovine sera samples were then separated into two, paired groups of preparations and labeled A and D, and B and C. Each preparation (prep) was subjected to a purification process, with the exception of prep C. Pre-immune injection purification of the A prep consisted of 50% purified by ammonium sulfate purification while the other 50% was purified via an affinity column containing the original Goat anti-bovine antibody to remove bovine proteins not specific to pregnant bovines. Pre-immune injection purification of the D prep consisted solely of the affinity column containing the original Goat anti-bovine antibody to remove bovine proteins not specific to pregnant bovines. Pre-immune injection purification of the B prep consisted of an ammonium sulfate purification.

The protocol used to generate the antibodies was the same for each of the four preps. On day 1, the rabbits were injected with 1 ml of one of the four respective preps mixed with Freunds complete adjuvant after 3-5 mls of pre-immune serum was collected. On day 21, each rabbit was given a second injection containing 1 ml of the respective antigen prep mixed with Freunds Incomplete Adjuvant. On day 30, each rabbit was test bled. On day 42, each rabbit was given a third injection containing 1 ml of the respective prep antigen mixed with incomplete Freunds Adjuvant. On day 50, each rabbit underwent a production bleed.

The blood from the production bleeds were all purified using Protein A and the serum resulting from prep B rabbits was further purified using ammonium sulphate precipitation.

### Example 3

This example describes the production of gold sols of different particle sizes and conjugation of antibody preparations to a gold sol for use in a method of detecting the pregnancy factor and thereby evaluating the maternal status of a test animal.

A. Direct Production of 20 nm Gold.

Approximately 100 ml of 20 nm gold can be prepared by mixing 97 ml of purified water heated to 60 C with 1 ml of 1% weight/volume tetrachloroauric acid (Aldrich). This mixture is stirred rapidly while 2.5 ml of 1% weight/volume sodium citrate (Sigma) is added. The resulting mixture is held at approximately 60 C for about 30 minutes until the color change has stopped. The solution is then boiled for about 15 minutes. The gold particles formed are about 20 nm in diameter and the maximum absorption of the solution is approximately 520 nm.

B. Direct Production of 40 nm Gold.

Approximately 100 ml of 40 nm gold can be prepared by mixing 97 ml of purified water heated to 95° C. with 1 ml of 1% weight/volume tetrachloroauric acid (Aldrich). This mixture is stirred rapidly while 2.5 ml of 1% weight/volume sodium citrate (Sigma) is added. The resulting mixture is held at approximately 95° C. for about 30 minutes until the color change has stopped. The solution is then boiled for about 30 minutes to use up any excess gold. The gold particles formed are about 40 nm in diameter and the maximum absorption of the solution is approximately 528 nm.

C. Indirect (Growth) Production of 40 nm Gold.

The 20 nm gold colloid prepared above is used as a seed to produce 140 ml of 40 nm gold. A solution of 120 ml of purified water and 20 ml of 20 nm gold colloid are stirred while heating to a boil. 1 ml of 1% weight/volume sodium citrate (Sigma) is added. A 0. 1% gold solution is prepared by adding 1.2 ml of a 1% gold solution (Aldrich) to 10.8 ml of purified water. 1.4 ml of 25 mM potassium carbonate is optionally added to this solution to control the pH. This 0.1% gold solution is added to the boiling gold colloid solution at a rate of 1 ml every minute. The colloid is boiled for at least 15 minutes after the final addition to ensure that all the gold has been reduced.

### Example 4

This example describes the conjugation of an antibody prep to a gold sol to produce an antibody conjugate for loading onto the testing device in the conjugate loading zone (the conjugate pad).

A. Salt Titration

A titration of the amount of antibody to be added to the gold colloid must be conducted with each antibody prep as different properties are important for each antibody prep used. To construct the titration, the pH of the colloid is adjusted to between about 6.5 and about 8.5 for an antibody with a 40 nm colloid or to between about 7 and about 9 for an antibody with a 20 nm colloid. The antibody is diluted to 0.1 mg/ml in a dilute buffer such as 5 mM borate or 5 mM phosphate. A series of ten tubes are set up containing 1 ml of gold colloid and 50 μl of antibody and/or water. The volume of antibody prep containing 0.1 mg/ml protein is increased in 5 μl increments from 0 μl up to 50 μl while the water added is correspondingly decreased in 5 μl increments from 50 μl down to 0 μl. Thus, the total volume of each tube remains constant at 1050 μl while the amount of antibody protein is serially diluted from 0.5 ug down to 0.0 ug across the ten tubes. After mixing each tube, 100 μl of 10% sodium chloride is added to each tube, mixed and incubated for 15 minutes. Unstabilized product has a blue or purple tinge while the stabilized product is red. The stabilization point in the titration curve is assumed to be the 1st tube that shows a very faint purple color.

B. Conjugation

The gold colloid is adjusted to the appropriate pH as determined by the assay above and slowly mixed with the corresponding volume of antibody prep. This protein/gold mixture is kept mixing for 30 minutes after which the pH is raised to greater than 9 with 0.1% NaOH. 10% bovine serum albumin is added dropwise to a final concentration of 0.1% and the conjugate is mixed for 15 minutes before the pH is reduced to 7.5 with 0.1% HCl. The resulting conjugate is stored in an appropriate solution at a higher concentration by first reducing the volume of the conjugate via any means known in the art (such as slow centrifugation of through a membrane-based concentration) and resuspending in either 50 mM phosphate pH 7.2, 0.1% BSA, 0.1% sodium azide or in 5 mM Borax pH 7.2, 0.1% BSA, 0.1% sodium azide at an optical density of about 5.

C. Testing of the Conjugate

The best testing method is a capture system on a lateral assay. To conduct such a test, nitrocellulose was cut into 2.5 cm wide strips and an absorbent wick 3MM paper paper with about 5 mm overlap between the nitrocellulose surface and the absorbent paper were used. A capture protein (such as goat anti-mouse IgG fab specific from Sigma) is striped onto the nitrocellulose at a concentration of 1 mg/ml and allowed to dry for 30 minutes. The gold conjugate is diluted in running buffer (50 mM phosphate pH 7.2, 0.5% BSA, 0.5% sodium azide) to about OD 0.5 at 520 nM. 40 μl of this solution is added into a well in a microtiter plate. The dried strip is cut into 0.5 cm wide strips and placed into each well containing conjugate. After each test has been allowed to run, the strips are compared visually.

### Example 5

This example describes accuracy testing of maternal status test based on each of the four antibody preps on blood from cows with confirmed pregnancy status based on palpation and/or ultra sound.

All antibody preps produced as described in Example 1 were conjugated with 40 nm Gold colloid as described in Example 2 and Example 3 to OD3 at pH 7.4. 101 vials of blood from cattle that had been artificially inseminated was obtained from a dairy. Sera was separated from each blood sample and each antibody was tested against each serum sample in a blind study as described in Example 3 above. About 400 tests were run in the United States and 40 tests were run in the United Kingdom. All of the cows were palpated and from 101 cows, only 33 were confirmed pregnant by palpation. At 30 days and at 40 days, a scan was done of the 21 test group cows.

From 21 cows 13 were confirmed pregnant by ultra sound scan. The accuracy results, broken down by the antibody preparation used in conducting the test, are shown in Table 1.

| Antibody Prep | Accuracy over all |
|---|---|
| Tested | tests conducted (%) |
| A | 91 |
| B | 83 |
| C | 87 |
| D | 93 |

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

What is claimed is:

1. A method for determining pregnancy of an animal comprising:
(a) obtaining a liquid biological sample of said animal; and
(b) testing for the presence of a pregnancy factor in said liquid biological sample.

2. The method of claim 1, wherein said animal is a bovine.

3. The method of claim 1, wherein said test comprises contacting said liquid biological sample to a pregnancy test device, wherein said test device comprises:
(a) a porous solid phase material capable of conveying a liquid sample in a fluid flow direction generally parallel to the length of said test device;
(b) a sample receiving zone within said porous solid phase material whereat the liquid sample and other assay reagents may be contacted with said device; and
(c) an antibody zone within said porous solid phase material comprising an immobilized antibody to said pregnancy factor of said animal disposed at a downstream location from said sample receiving zone.

4. The method of claim 3, wherein said porous solid phase material further comprises a labeled antibody zone disposed at a downstream location from said sample receiving zone and upstream from said antibody zone.

5. The method of claim 4, wherein said labeled antibody comprises a particle composition, wherein said particle composition comprises:
(a) a sol particle; and
(b) an antibody to said pregnancy factor of said animal conjugated thereto.

6. The method of claim 5, wherein said sol particle is gold.

7. The method of claim 6, wherein said gold sol particle has a particle size of from about 15 nm to about 100 nm.

8. The method of claim 7, wherein said gold sol particle has a particle size of about 40 nm.

9. The method of claim 5, wherein said antibody is isolated by a process comprising:
(a) injecting a biological fluid of said animal to a second animal, wherein said second animal is capable of producing antibody to said biological fluid, and wherein said biological fluid comprises said pregnancy factor of said animal;
(b) isolating antibodies from said second animal; and
(c) removing antibodies that are negative to said pregnancy factor of said animal.

10. The method of claim 9, wherein said animal is a bovine which is pregnant for from about 20 days to about 40 days.

11. The method of claim 9, wherein said biological fluid is selected from the group consisting of whole blood, serum, chorionic fluid, urine, saliva, milk, perspiration and combinations thereof.

12. The method of claim 11, wherein said biological fluid is serum.

13. The method of claim 1, wherein said liquid biological sample is contacted with a particle composition prior to said step (b), wherein said particle composition comprises:
(a) a sol particle; and
(b) an antibody to said pregnancy factor of said animal conjugated thereto.

14. The method of claim 13, wherein said sol particle is gold having a particle size of from about 15 nm to about 100 nm.

15. The method of claim 13, wherein said sol particle is latex.

16. The method of claim 13, wherein said antibody is isolated by a process comprising:
(a) injecting a biological fluid of said animal to a second animal, wherein said second animal is capable of producing antibody to said biological fluid, and wherein said biological fluid comprises said pregnancy factor of said animal;
(b) isolating antibodies from said second animal; and
(c) removing antibodies that are negative to said pregnancy factor of said animal.

17. The method of claim 16, wherein said animal is a bovine which is pregnant for from about 20 days to about 40 days.

18. The method of claim 16, wherein said biological fluid is selected from the group consisting of serum, urine, saliva, milk, perspiration and combinations thereof.

19. A method for isolating an antibody to an pregnancy factor of an animal comprising:
(a) injecting a biological fluid of a pregnant animal to a second animal, wherein said second animal is capable of producing antibody to said biological fluid, and wherein said biological fluid comprises said pregnancy factor of said animal;
(b) isolating antibodies from said second animal; and
(c) removing antibodies that are negative to said pregnancy factor of said animal.

20. The method of claim 19, wherein said animal is a bovine.

21. The method of claim 20, wherein said bovine is pregnant for less than about 100 days.

22. The method of claim 20, wherein said bovine is pregnant for from about 20 days to about 40 days.

23. The method of claim 19, wherein said liquid biological sample is selected from the group consisting of serum, urine, saliva, milk, perspiration and combinations thereof.

24. The method of claim 19, wherein said liquid biological sample is serum.

25. The method of claim 19, wherein said second animal is selected from the group consisting of goat, rabbit, poultry, sheep, swine, feline, horse, donkey, ass, rodents, and dog.

26. The antibody produced by the process comprising:
(a) isolating body fluid from an animal at a selected stage of pregnancy;
(b) partially purifying said body fluid;
(c) injecting said partially purified body fluid into a host animal of a different species;
(d) removing blood from said host animal; and,
(e) partially purifying said blood to isolate antibodies from said host animal blood.

27. The antibody of claim 26 wherein said biological fluid is selected from the group consisting of blood, serum, perspiration, saliva, and milk.

28. The antibody of claim 26 wherein said partial purification comprises purification over an affinity column.

29. The antibody of claim 28 wherein said affinity column comprises a goat anti-bovine IgG affinity column.

30. The antibody of claim 26 wherein said partial purifying step (e) comprises Protein A purification.

31. The antibody produced by the process comprising:
(a) isolating serum from a cow that is pregnant;
(b) purifying said serum over a goat anti-bovine IgG affinity column;
(c) injecting said purified serum into a non-bovine animal;
(c) removing blood from said non-bovine animal; and,
(d) partially purifying said blood with Protein A to isolate antibody from said non-bovine blood.

32. The antibody produced by the process comprising:
(a) isolating serum from a horse that is pregnant;
(b) partially purifying said serum;
(c) injecting said purified serum into a non-bovine animal;
(d) removing blood from said non-equine animal; and,
(e) partially purifying said blood to isolate antibody from said non-equine blood.

33. A method for determining pregnancy of an animal comprising:
(a) obtaining a liquid biological sample of said animal; and,
(b) testing for the presence of a pregnancy factor in said liquid biological sample by particle agglutination immunoassay.
